(19) United States
(12) Patent Application Publication
(10) Pub. No.: US 2008/0263749 A1
(43) Pub. Date: Oct. 30, 2008

(54) BANDLESS HEARING PROTECTION MUFFS

(75) Inventors: Waihong Leong, Roswell, GA (US); Rick Purcell, Alpharetta, GA (US); James Blaine Wolford, Chicago, IL (US); Scott Madison Belliveau, Plainfield, IL (US); Aleksey Pirkhalo, Chicago, IL (US); Sean S. Corbin, Morton Grove, IL (US); Ki Bok Song, Des Plaines, IL (US)

Correspondence Address: Christopher M. Goff (27839);ARMSTRONG TEASDALE LLP, ONE METROPOLITAN SQUARE, SUITE 2600, ST. LOUIS, MO, 63102, US

(73) Assignee: KIMBERLY-CLARK WORLDWIDE, INC., Neenah, WI (US)

(21) Appl. No.: 11/742,492

(22) Filed: Apr. 30, 2007

(51) Int. Cl. A42B 1/06; A61F 11/06

(52) U.S. Cl. 2/209; 381/72

(57) ABSTRACT

The present disclosure generally relates to devices for covering an ear, and more particularly to such devices that are self-attaching and used for sound optimization, such as noise reduction. More specifically, the present disclosure is directed to hearing protection muffs that comprise a sound attenuating cap, a self-attachment member for engaging the sound attenuating cap to the ear of a user, and optionally a sealing member. Advantageously, the muffs of the present disclosure have good sound attenuating properties and do not require a band or other external attachment device to secure the muffs to the ear.

### BACKGROUND OF DISCLOSURE

The present disclosure generally relates to devices for covering an ear, and more particularly to such devices that are self-attaching and used for sound optimization, such as noise reduction.

The need for adequate hearing protection in high noise environments has long been recognized among those concerned with health and safety issues, and much effort has gone into providing such protection. However, most experts in this field would acknowledge that this effort has not been very successful. Protective devices have proliferated yet remained mediocre in performance. Workers in high noise environments who should use these devices often do not, or use them only under duress from their employers. Individuals that work in high noise environments rarely understand that the effects of high noise exposure are not limited to the moment, but are cumulative as well.

Comfort is the primary barrier to the effective use of hearing protectors in the work place. Conventional hearing protectors such as earplugs and ear muffs are typically very uncomfortable to wear, resulting in a low compliance of wearing hearing protectors in work environments that require hearing protection.

For example, existing disposable foam ear plugs are uncomfortable for wearers with small ear canals, are difficult to properly insert, and/or cause discomfort by creating a vacuum in the ear canal during removal. Existing disposable foam ear plugs require the user to compress the area of the plug and insert it into the ear canal where it then attempts to re-expand. This method can cause discomfort for people with small ear canals in that the more compressed the ear plug, the greater its exerted force towards re-expansion. Further, existing disposable foam ear plugs require the user to roll the foam between their fingers to compress the foam to a sufficient area for proper insertion. If this step is not done, or is insufficiently done, the ear plug is often inserted improperly so as to not provide optimal protection. Also, if the user has dirty hands when compressing the ear plug, dirt and/or germs are then put into the ear canal with the inserted ear plug.

Comfort is also a problem with conventional ear muffs. For instance, conventional ear muffs are typically held in place over the ear through the use of a band or other external device. However, the use of such external device can exert pressure on the head of the user and become uncomfortable when worn for prolonged periods of time. Additionally, conventional ear muffs are often cumbersome because of their size and weight, due to the large amounts of oftentimes dense materials needed to sufficiently block sound from entering the ears.

Accordingly, while various types of ear protection devices exist, there remains a clear need for an ear protection device that is more comfortable than currently available devices when used for extended periods of time, but that provides good sound attenuating effects.

### SUMMARY OF THE DISCLOSURE

The present disclosure generally relates to devices for covering an ear, and more particularly to such devices that are self-attaching and used for sound optimization, such as noise reduction. More specifically, the present disclosure is directed to hearing protection muffs that comprise a sound attenuating cap, a self-attachment member for engaging the sound attenuating cap to the ear of a user, and optionally a sealing member. Advantageously, the muffs of the present disclosure have good sound attenuating properties and do not require a band or other external attachment device to secure the muffs to the ear. Consequently, the muffs avoid many of the disadvantages associated with banded earmuffs.

In one aspect, the present disclosure is directed to a bandless, hearing protection muff. The muff comprises a sound attenuating cap defining a cavity for covering an external ear of a user, the sound attenuating cap having a rim portion for encircling the ear; a sealing member having a surface for being placed against a portion of the user's head; and a self-attachment member for engaging the sound attenuating cap to the ear of the user. The sound attenuating cap comprises an outer layer comprising a sound reflecting material, the sound reflecting material having a sound absorption coefficient of no more than about 0.4 at a frequency of from about 800 Hz to about 10,000 Hz, and an inner layer disposed adjacent to the outer layer, the inner layer comprising a sound absorbing material, the sound absorbing material having a sound absorption coefficient of at least about 0.6 at a frequency of from about 800 Hz to about 10,000 Hz. The sound attenuating cap attenuates sound at from about 8 dB to about 33 dB over the frequency range of from about 125 Hz to about 8,000 Hz.

In another aspect, the present disclosure is directed to a bandless, hearing protection muff comprising a sound attenuating cap defining a cavity for covering an external ear of a user, the sound attenuating cap having a rim portion for encircling the ear; a self-attachment member for engaging the sound attenuating cap to the ear of the user, the self-attachment member comprising a substantially U-shaped ear clip providing a first end portion and a second end portion spaced from the first end portion, and a rigid frame comprising a hinge, the hinge comprising a torsion spring, the rigid frame being disposed adjacent to the rim portion of the sound attenuating cap, wherein the first end portion of the ear clip extends through the hinge, and the second end portion of the ear clip is capable of wrapping behind the pinna of the ear of the user; and a sealing member disposed adjacent to the rigid frame, the sealing member having a surface for being placed against a portion of the user's head. The sound attenuating cap comprises an outer layer comprising a sound reflecting material, the sound reflecting material having a sound absorption coefficient of no more than about 0.4 at a frequency of from about 800 Hz to about 10,000 Hz, and an inner layer disposed adjacent to the outer layer, the inner layer comprising a sound absorbing material, the sound absorbing material having a sound absorption coefficient of at least about 0.6 at a frequency of from about 800 Hz to about 10,000 Hz. The sound attenuating cap attenuates sound at from about 8 dB to about 33 dB over the frequency range of from about 125 Hz to about 8,000 Hz.

In another aspect, the present disclosure is directed to a bandless, hearing protection muff. The muff comprises a sound attenuating cap for covering an external ear of a user; and a self-attachment member for engaging the sound attenuating cap to the ear of the user. The self-attachment member comprises an ear clip comprising a first end portion and a second end portion spaced from the first end portion, a hinge for attaching the ear clip to the sound attenuating cap, and a snapping mechanism. The sound attenuating cap comprises an outer layer comprising a sound reflecting material, the sound reflecting material having a sound absorption coefficient of no more than about 0.4 at a frequency of from about 800 Hz to about 10,000 Hz, and an inner layer disposed adjacent to the outer layer, the inner layer comprising a sound absorbing material, the sound absorbing material having a sound absorption coefficient of at least about 0.6 at a frequency of from about 800 Hz to about 10,000 Hz. The sound attenuating cap attenuates sound at from about 8 dB to about 33 dB over the frequency range of from about 125 Hz to about 8,000 Hz.

In still another aspect, the present disclosure is directed to a bandless, hearing protection muff. The muff comprises a sound attenuating cap for covering an external ear of a user; and a self-attachment member for engaging the sound attenuating cap to the ear of the user. The self-attachment member comprises an ear clip comprising a first end portion and a second end portion spaced from the first end portion; and a hinge for attaching the ear clip to the sound attenuating cap, the hinge comprising a torsion spring. The sound attenuating cap comprises an outer layer comprising a sound reflecting material, the sound reflecting material having a sound absorption coefficient of no more than about 0.4 at a frequency of from about 800 Hz to about 10,000 Hz, and an inner layer disposed adjacent to the outer layer, the inner layer comprising a sound absorbing material, the sound absorbing material having a sound absorption coefficient of at least about 0.6 at a frequency of from about 800 Hz to about 10,000 Hz. The sound attenuating cap attenuates sound at from about 8 dB to about 33 dB over the frequency range of from about 125 Hz to about 8,000 Hz.

Other objects and features will be in part apparent and in part pointed out hereinafter.

### BRIEF DESCRIPTION OF THE DRAWINGS

FIG. 1 is a cross-sectional view of a muff of the present disclosure attached to the ear of a user.

FIG. 2 is a depiction of a muff of the present disclosure.

FIG. 3 is a frontal view of the rim portion of a sound attenuating cap of the present disclosure.

FIG. 4 is a depiction of a self-attachment member of the present disclosure.

FIG. 5 is a depiction of a muff of the present disclosure.

FIG. 6 is a bottom-view of the hinge depicted in FIG. 5.

FIG. 7 is a depiction of a muff of the present disclosure.

FIG. 8 is a rear view of the ear clip and ear pad depicted in FIG. 7.

FIGS. 9A, 9B, and 9C depict ear pads suitable for use with the self-attachment member depicted in FIG. 7.

FIG. 10 is a frontal view of the rim portion of a sound attenuating cap with a self-attachment member of the present disclosure.

FIG. 11 is a cross-sectional view of the muff depicted in FIG. 12.

FIG. 12 is a depiction of a muff of the present disclosure.

FIG. 13 is a frontal view of the rim portion of a sound attenuating cap with a self attachment member of the present disclosure.

FIG. 14 is a depiction of a muff of the present disclosure.

FIG. 15 is a cross-sectional view of the muff depicted in FIG. 16.

FIG. 16 is a depiction of a muff of the present disclosure.

FIG. 17 is a depiction of a muff of the present disclosure.

FIG. 18 is a cross-sectional view of the muff depicted in FIG. 17.

FIG. 19 is a depiction of a muff of the present disclosure.

FIG. 20 shows the muff depicted in FIG. 19 in the closed position.

Corresponding reference characters indicate corresponding parts throughout the drawings.

### DETAILED DESCRIPTION OF THE DISCLOSURE

The present disclosure generally relates to devices for covering an ear, and more particularly to such devices that are self-attaching and used for sound optimization, such as noise reduction. More specifically, the present disclosure is directed to hearing protection muffs that comprise a sound attenuating cap, a self-attachment member for engaging the sound attenuating cap to the ear of a user, and optionally a sealing member. Advantageously, the muffs of the present disclosure have good sound attenuating properties and do not require a band or other external attachment device to secure the muffs to the ear. Consequently, the muffs avoid many of the disadvantages associated with banded earmuffs.

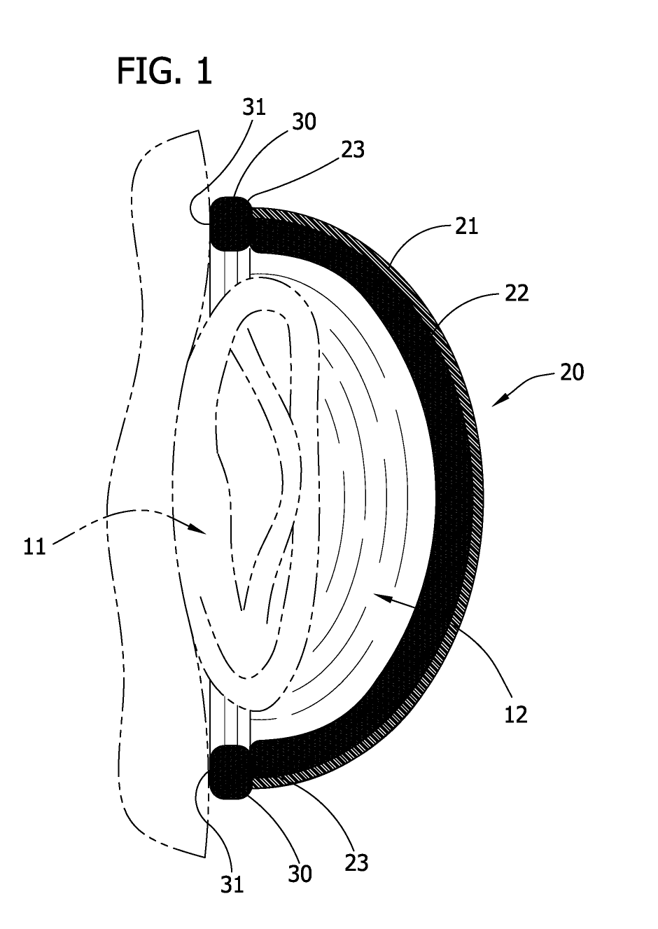

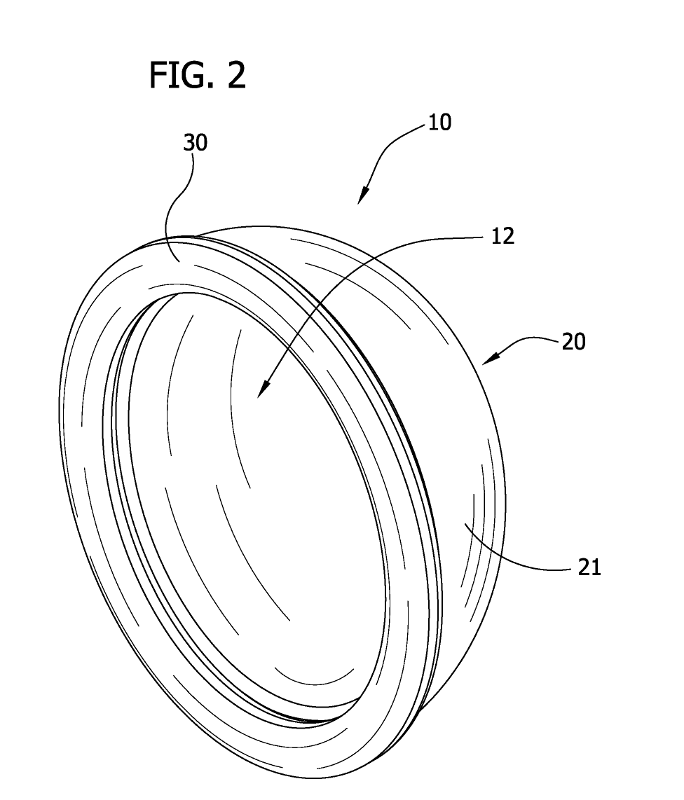

Referring now to the drawings and in particular FIGS. 1 and 2, for example, there is depicted a hearing protection muff 10 according to the present disclosure. The hearing protection muff 10 comprises a sound attenuating cap 20 defining a generally oval or circular shaped cavity 12 for covering an external ear 11. The sound attenuating cap 20 may be domed, as illustrated in the Figures, flat (not shown), or any other suitable shape. A sealing member 30 is disposed adjacent to the sound attenuating cap 20, encircling the ear 11. The sealing member 30 has a surface 31 for being placed against a portion of the user's head.

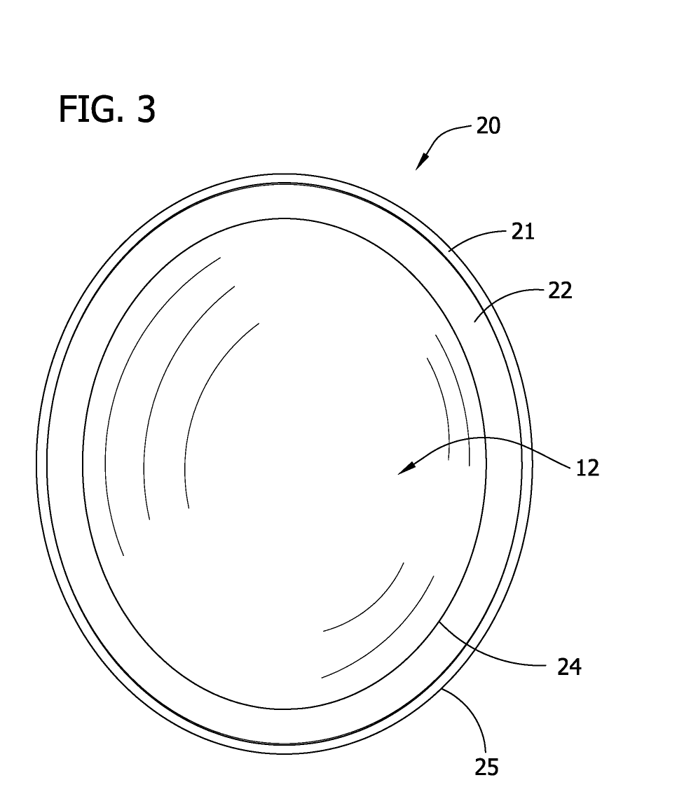

The sound attenuating cap 20, illustrated for example in FIGS. 1-3, comprises an outer layer 21 that comprises a sound reflecting material, and an inner layer 22 that comprises a sound absorbing material. In one embodiment, the inner layer 22 forms the inner surface of the hearing protection muff 10 that faces the ear 11 of the user. The inner layer 22 is disposed adjacent to the outer layer 21 and is uniformly bonded thereto, for example, using adhesives or any other suitable means. The outer layer 21 forms the outer surface of the sound attenuating cap 20 that faces away from the ear of the user.

The sound attenuating cap 20 defines a generally oval or circular shaped cavity 12 for covering an external ear 11. The edges of the outer layer 21 and inner layer 22 of the sound attenuating cap 20 terminate in a rim portion 23, which encircles the ear 11. The rim portion 23 has a thickness defined by an inner circumference 24, which runs along the interior side (cavity-facing side) of the inner layer 22, and an outer circumference 25, which runs along the outer surface of the outer layer 21. In an alternate embodiment, the outer layer 21 may extend around the rim portion 23, covering all or a portion of the inner layer 22 along the rim portion.

The outer layer 21 and inner layer 22 of the sound attenuating cap 20 advantageously work in combination to attenuate sound. More specifically, the outer layer 21 of the sound attenuating cap 20 comprises a sound reflecting material that reflects sound away from the cap, while the inner layer 22 of the sound attenuating cap 20 comprises a sound absorbing material for absorbing sound that penetrates through the outer layer 21. By using a combination of sound reflecting and sound absorbing materials in the sound attenuating cap, sound energy that is not reflected by the outer layer 21 can be absorbed by the inner layer 22 of the sound attenuating cap, thus providing for an improved sound attenuating effect.

Typically, the outer layer 21 and the inner layer 22 of the sound attenuating cap 20 cooperate to attenuate at least about 8 decibels (dB) of sound over the frequency range of from about 125 Hz to about 8,000 Hz. Preferably, the outer layer and the inner layer of the sound attenuating cap cooperate to attenuate sound at from about 8 dB to about 33 dB, and more preferably at from about 17 dB to about 30 dB over the frequency range of from about 125 Hz to about 8,000 Hz. In one embodiment, the sound attenuating cap 20 preferably has an overall Noise Reduction Rating of at least about 8 dB, more preferably from about 17 dB to about 30 dB.

The efficiency at attenuating sound of the sound reflecting and sound absorbing materials used to form the sound attenuating cap can be expressed in terms of their sound absorption coefficient. The sound absorption coefficient of a material can have a value between 0 and 1, with 0 representing no sound absorption and 100% sound reflection, and 1 representing 100% sound absorption and no sound reflection. The sound absorption coefficient can be expressed as: α=Iₐ/Iᵢ, wherein Iₐ is the sound intensity absorbed (Watts per square meter (W/m²)) and Iᵢ is the incident sound intensity (W/m²). Methods for measuring sound absorption coefficients of materials are known and include, for example, ASTM E1050 “Standard Test Method for Impedance and Absorption of Acoustical Materials Using a Tube, Two Microphones and a Digital Frequency Analysis System.” While the sound absorption coefficient of a material may vary with the frequency of the sound, advantageously, the sound reflecting materials of the outer layer are capable of reflecting both high and low frequency sound, while the sound absorbing materials of the inner layer are capable of absorbing both high and low frequency sound.

A variety of sound reflecting materials may be used in the outer layer. Preferably, the sound reflecting materials that make up the outer layer will reflect at least about 60%, more preferably at least about 80% of sound energy over the frequency range of from about 800 Hz to about 10,000 Hz, thus having a sound absorption coefficient of no more than about 0.4 and more preferably no more than about 0.2 over the frequency range of from about 800 Hz to about 10,000 Hz. Examples of suitable sound reflecting materials include, for example, mass loaded vinyl, high density plastic, metals, woods, and combinations thereof. Specific examples of suitable plastics include polycarbonate, high density polyethylene, and polyvinylidence chloride, and the like. Specific examples of metals include lead, steel, brass, bronze, and the like. Specific examples of woods include birch, oak, larch, and the like. Preferably, the outer layer 21 of the sound attenuating cap 20 comprises mass loaded vinyl.

As noted above, the inner layer 22 comprises sound absorbing materials that advantageously act to absorb sound that penetrates through the outer layer 21, thus improving the sound attenuating effects of the sound attenuating cap 20. A variety of sound absorbing materials may be used in the inner layer 22. Preferably, the sound absorbing materials that make up the inner layer 22 will absorb at least about 60% and more preferably at least about 80% of sound energy over the frequency range of from about 800 Hz to about 10,000 Hz, thus having a sound absorption coefficient of at least about 0.6 and more preferably at least about 0.8 over the frequency range of from about 800 Hz to about 10,000 Hz. Examples of suitable sound absorbing materials include, for example, glass fibers, fibrous mineral wool, open cell foam, semi-open cell foam, and the like. Other suitable sound absorbing materials include polyolefin fibers, polyurethane foams, and the like.

It should be understood that the choice of sound absorbing materials for use in the inner layer 22 (and the desired sound absorption coefficient) may vary depending on the type of sound reflecting material used in the outer layer 21, but is preferably selected so that the sound attenuating cap (i.e., the combination of the outer layer 21 and the inner layer 22 of the sound attenuating cap 20) attenuates from about 8 dB to about 33 dB over the frequency range of from about 125 Hz to about 8,000 Hz.

In addition to sound attenuating capability, other factors that may be considered in selecting the combination of materials for the inner and outer layers of the sound attenuating cap include the desired weight and size of the sound attenuating cap, as well as cost of making the cap. For instance, depending on the type of material selected to form the outer layer 21 and the inner layer 22, the thickness of the sound attenuating cap may vary, thus affecting the overall size and weight of the sound attenuating cap 20. In one embodiment, the outer layer 21 typically has a thickness of at least about 1 millimeter, and more typically has a thickness of from about 1 millimeter to about 10 millimeters, preferably from about 2 millimeters to about 5 millimeters. The inner layer 22 typically has a thickness of at least about 5 millimeters, and more typically has a thickness of from about 5 millimeters to about 40 millimeters, preferably from about 10 millimeters to about 20 millimeters. Preferably, the materials used to form the outer layer 21 and inner layer 22 are selected so that the thickness of the sound attenuating cap 20 (e.g., along the rim portion 23) is from about 15 millimeters to about 30 millimeters. A sound attenuating cap having such characteristics should have good sound attenuating properties, yet still be relatively light in weight, improving comfort when the muff is worn.

As can be seen with reference again to FIGS. 1 and 2, the hearing protection muff 10 of the present disclosure may further comprise a sealing member 30. The sealing member 30 has a surface 31 for being placed against a portion of the user's head. More specifically, the sealing member 30 provides a seal between the sound attenuating cap 20 and the surface around the ear which contacts the muff 10. Advantageously, the presence of the sealing member 30 may reduce the amount of sound energy that penetrates between the edges of the muff and the user's head when the muff is worn. The sealing member 30 may also act as a cushion, providing a softer and more comfortable surface for being placed against a portion of the user's head when the muff is in use.

The placement of the sealing member 30 may vary, depending on the specific design of the muff 10. Preferably, the sealing member 30 is positioned such that a surface 31 of the sealing member 30 will be placed against a portion of the user's head when the muff 10 is worn.

Typically, the sealing member 30 is disposed adjacent to the rim portion 23 of the sound attenuating cap 20. In other embodiments, however, the sealing member 30 may be disposed adjacent to all or a part of the self-attachment member, as described elsewhere herein. The sealing member 30 may be disposed adjacent to the entirety of the rim portion 23 (e.g., it may encircle the entire opening of cavity 12), or alternately, may be disposed adjacent to any fraction of the rim portion 23. For example, in one embodiment, the sealing member 30 is disposed adjacent to the lower one-half to one-third of the rim portion 23. This embodiment may be advantageous in that it allows safety eyewear to be worn in combination with the muff without interfering with the sealing member 30 (e.g., by breaking the sealing member's contact with the user's head along the temple).

The sealing member 30 may be attached to the sound attenuating cap 20 using any suitable means, such as, for example, adhesives. Examples of suitable adhesives include silicone adhesives, hydrogel adhesives, and the like.

In certain embodiments, the sealing member 30 may comprise a cushioning material, such as a polyurethane foam with vinyl skin, ethylene-vinyl acetate, silicone rubber, ethylene propylene rubber, and the like. Alternately or in addition, the sealing member 30 may comprise an adhesive material, such as, for example, a contact adhesive, such as a pressure-sensitive adhesive suitable for long-term skin contact, a silicone adhesive, a hydrogel adhesive, and the like.

In certain embodiments, the muff 10 may further comprise a layer of adhesive disposed on the surface 31 of the sealing member 30 that contacts the head of the user. This layer of adhesive helps to adhere the muff to the user's ear, and provides a better acoustic seal to minimize sound leaking into the cap. Examples of suitable adhesives include, for example, silicone adhesives, hydrogel adhesives, pressure sensitive adhesives, and the like.

Advantageously, the hearing protection muffs of the present disclosure further comprise a self-attachment member. As discussed above, conventional earmuffs are typically held in place about the ear of a user with a band or other external device. However, bands or other external attachment devices can exert pressure on the head of a user, which is oftentimes uncomfortable, especially when the earmuffs are worn for an extended period of time. Advantageously, the hearing protection muffs of the present disclosure comprises a self-attachment member that requires no band or other external device for maintaining the engagement of the hearing protection muff with the ear of the user, thus avoiding problems associated with banded earmuffs.

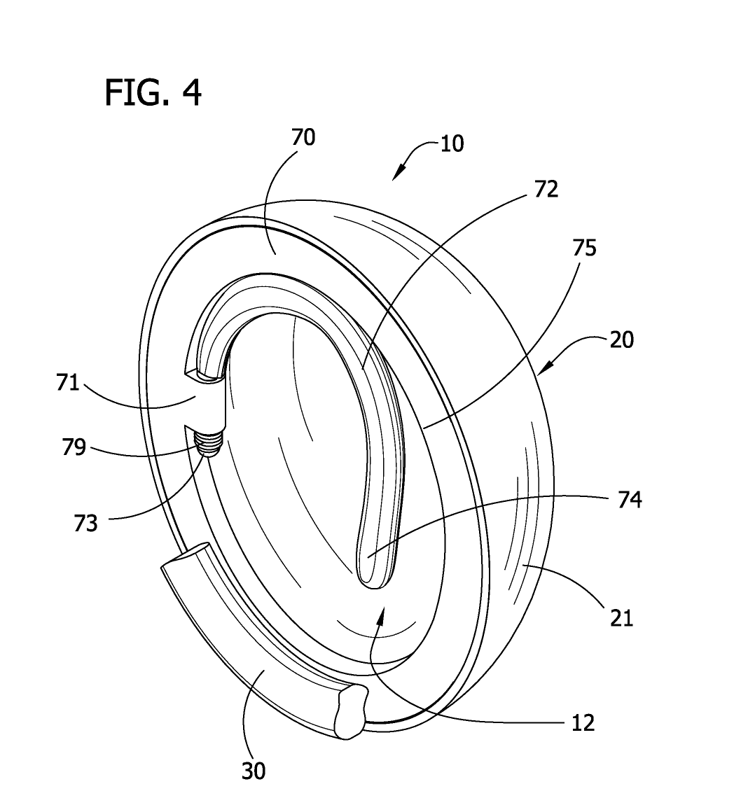

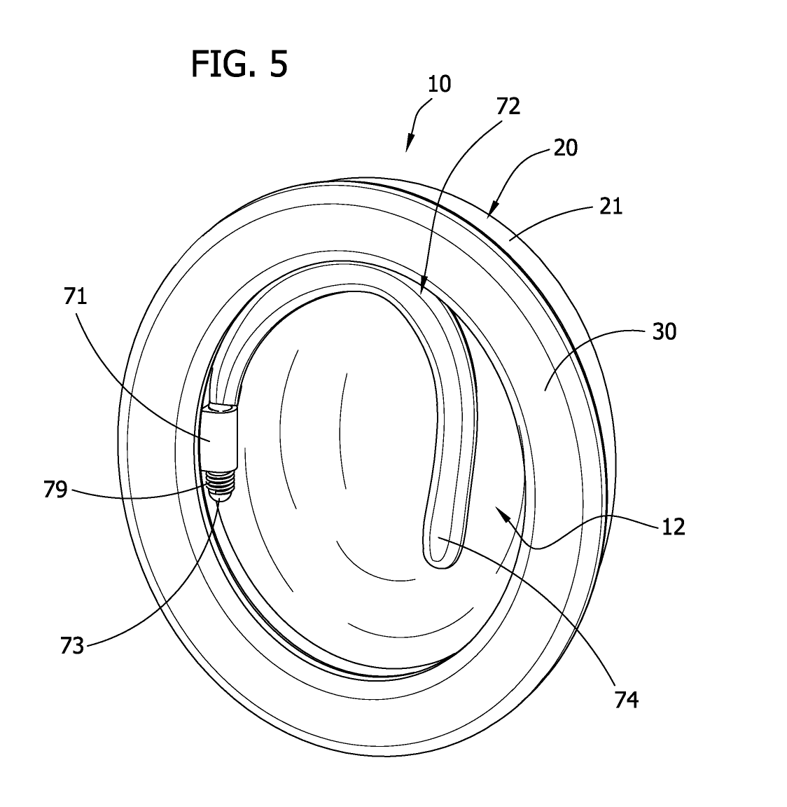

A variety of suitable self-attachment members may be used in combination with the muffs of the present disclosure, some of which are depicted in FIGS. 4-20, hereinafter described.

One preferred example of a self-attachment member is illustrated in FIGS. 4-6, which depict a hearing protection muff 10 comprising a self-attachment member that comprises a substantially U-shaped ear clip 72 and a rigid frame 70 comprising a hinge 71. The ear clip 72 comprises a first end portion (i.e., an “attached” end portion) 73 and a second end portion (i.e., a “free” end portion) 74 spaced from the first end portion to form a substantially “U” shape. The first end portion 73 of the ear clip 72 extends through the hinge 71, and the second end portion 74 of the ear clip 72 is capable of wrapping behind the pinna of the ear of the user. It should be understood that the exact shape of the ear clip 72 and the distance between the first end portion 73 and the second end portion 74 is not critical, so long as the shape and dimensions of the ear clip are such that the second (“free”) end portion 74 can fit behind the pinna of the user to hold the muff 10 in place over the ear.

Typically, the ear clip 72 is formed from a flexible material (i.e., a material having a degree of bendability). The ear clip 72 is preferably rigid enough to maintain its position behind the ear of the user when the muff 10 is being worn, but not so rigid that use of the ear clip 72 is uncomfortable for the user. Examples of suitable materials from which the ear clip 72 may be made include, for example, plastics such as polypropylene, polyvinyl chloride, and polycarbonate; metals such as titanium, steel, and aluminum composites; and elastomers such as silicon, thermoplastic elastomers, polyurethane rubber, and ethylene propylene rubber, and the like. In certain embodiments, the free end portion 74 of the ear clip 72 may be contoured to conform to the shape of the pinna, to increase comfort to the user when the free end portion 74 of the ear clip 72 is wrapped behind the ear of the user.

As noted above, the ear clip 72 is attached to the sound attenuating cap 20 by means of a rigid frame 70 comprising a hinge 71. The rigid frame 70 is disposed adjacent to the rim portion 23 of the sound attenuating cap 20, and may extend around the entire circumference of the cavity 12, as depicted in FIG. 4. The rigid frame 70 may be disposed adjacent to the entire surface of the rim portion 23, or alternately, may cover only a portion of the rim portion 23. The rigid frame 70 is attached to the rim portion 23 of the sound attenuating cap 20 using any suitable means, such as, for example, using adhesives.

The rigid frame 70 provides support for the hinge 71 and ear clip 72, and therefore, is preferably made of materials that are relatively inflexible and that are capable of maintaining their shape, even when force is applied (for example, when the muff 10 is being attached, as described hereinafter). Examples of suitable materials from which the rigid frame 70 can be made include, for example, plastics such as polypropylene, polyvinyl chloride, polycarbonates, and the like; and metals, such as titanium, steel, aluminum composites, and the like. In one particular embodiment, the rigid frame 70 may be an extension of the outer layer 21 of the sound attenuating cap 20. For example, the outer layer 21 of the sound attenuating cap 20 may extend around the rim portion (not shown), covering all or a portion of the inner layer along the rim portion, and forming the rigid frame 70. Thus in this embodiment, the rigid frame 70 may comprise sound reflecting materials, such as those described above as suitable for formation of the outer layer 21.

The hinge 71 is connected to the rigid frame 70, and generally is formed of the same material used to make the rigid frame 70. The hinge 71 extends inwardly from the inner edge 75 of the rigid frame 70, as depicted in FIG. 4. The exact location of the hinge 71 along the inner edge 75 of the rigid frame 70 is not critical, so long as it is in a location that allows the free end portion 74 of the ear clip 72 to wrap around the pinna of the ear of a user to secure the muff 10 to the ear.

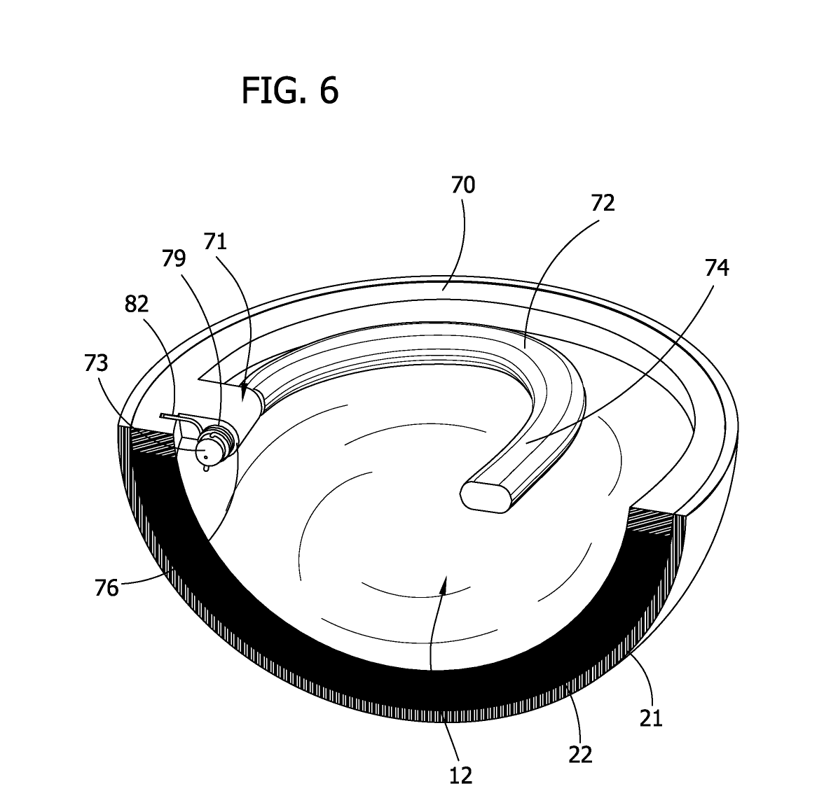

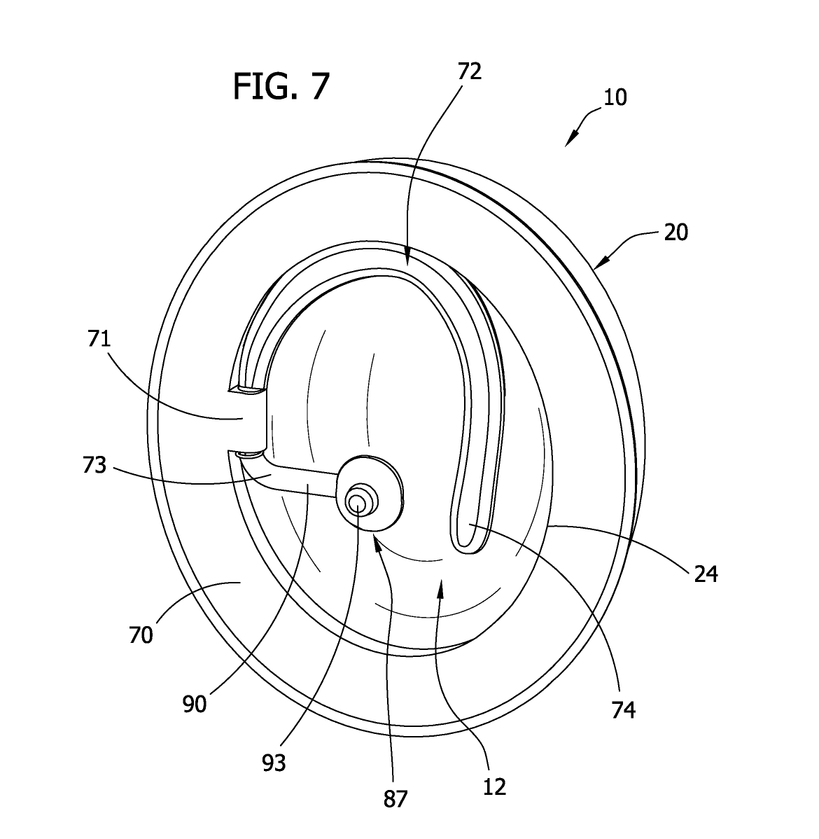

As noted above, the first end portion 73 of the ear clip 72 extends through the hinge 71, which mounts the ear clip 72 to the rigid frame 70. Referring now to FIG. 6, the hinge 71 comprises a torsion spring 79, which wraps around the first end portion 73 of the ear clip 72. A free arm 82 of the torsion spring 79 hooks around the rigid frame 70, as depicted in FIG. 6. The torsion spring 79 is thus oriented to resist outward movement of the second end portion 74 of the ear clip 72 away from the cavity 12, which operates to clip the muff 10 to the ear of a user. It is to be understood that the hinge configuration depicted in FIG. 6 is illustrative, and other hinge configurations may also be suitably used. It should further be understood that although the torsion spring 79 is depicted in FIGS. 4-6 as outside the hinge casing 76, in other embodiments, the torsion spring 79 may be located within the hinge casing 76 (see, e.g., FIG. 7, as described elsewhere herein).

Referring again to FIGS. 4-6, to engage the muff 10 to the ear of the user, the user applies force to the second (“free”) end portion 74 of the ear clip 72 and progressively pivots it outward away from the cavity 12. This movement compresses the torsion spring 79. During this initial movement, the torsion spring 79 resists the pivoting movement of the ear clip 72, so if the force being applied to the ear clip 72 is released, the torsion spring 79 urges the ear clip back to its original “at rest” position, i.e., generally parallel to the plane defined by the rigid frame 70. Once the second end portion 74 of the ear clip 72 has been pivoted outward, the second end portion 74 of the ear clip 72 can be wrapped behind the pinna of the ear of the user (typically over the top portion of the pinna). As noted above, when the force applied by the user to the second end portion 74 of the ear clip 72 is released, the torsion spring 79 urges the ear clip back to its original “at rest” position. This causes the second end portion 74 of the ear clip 72 to push against the back of the pinna of the ear of the user, thus holding the muff 10 in place over the ear.

As noted above, the muff 10 may further comprise a sealing member 30, which may be disposed adjacent to the rigid frame 70. The sealing member 30 may overlie all or a portion of the rigid frame 70. Preferably, the sealing member 30 overlies the entirety of the rigid frame 70, to provide a comfortable fit of the muff 10 against the head of the user.

Another preferred example of a self-attachment member is illustrated in FIGS. 7-9. Referring now to FIG. 7, there is depicted a hearing protection muff 10 comprising a self-attachment member that comprises a substantially U-shaped ear clip 72, a rigid frame 70 comprising a hinge 71, and an ear pad 87. The self-attachment member of this embodiment is substantially the same as that illustrated in FIGS. 4-6, except there is extending from the first end portion 73 of the ear clip 72 an extension member 90 with an ear pad 87 attached thereto. More particularly, the extension member 90 extends inwardly from the end of the first end portion 73 of the ear clip 72 towards the center of the cavity 12, generally parallel to the plane defined by the rigid frame 70. The ear pad 87 is attached to the extension member 90. The extension member 90 may extend inwardly from the first end portion 73 of the ear clip 72 generally far enough so that the ear pad 87 is positioned over the external auditory meatus when the muff 10 is worn. The ear pad 87 thus positioned acts to block out sound, improving the sound attenuating effect of the muff 10. In an alternate embodiment, the extension member 90 with attached ear pad 87 may be positioned such that the ear pad 87 overlies the tragus of a user's ear, so that pressure exerted by the ear pad 87 when the muff 10 is worn will fold the tragus over the auditory canal of the user's ear to block out sound without the necessity of the ear pad 87 entering the auditory canal.

There are several advantages to including ear pad 87 in the muff design. For example, the ear pad 87 may act as a secondary acoustic seal, improving the sound attenuating effects of the muff. In certain embodiments where the muff comprises an ear pad, the sound attenuating cap 20, hereinbefore described, may actually be designed with a reduced mass of sound reflecting and sound absorbing materials, resulting in a lighter weight muff, with the muff still achieving the same noise reduction effects of higher mass caps that do not have an ear pad. Additionally, the ear pad 87 provides additional contact area of the self-attachment member with the ear of the user, thus helping to secure the muff 10 in place over the ear.

The ear pad 87 may comprise an ear plug or other resilient member. Preferably, the ear pad is comprised of a resilient material such as, for example, foamed rubber, plastic, gel, silicone, and the like.

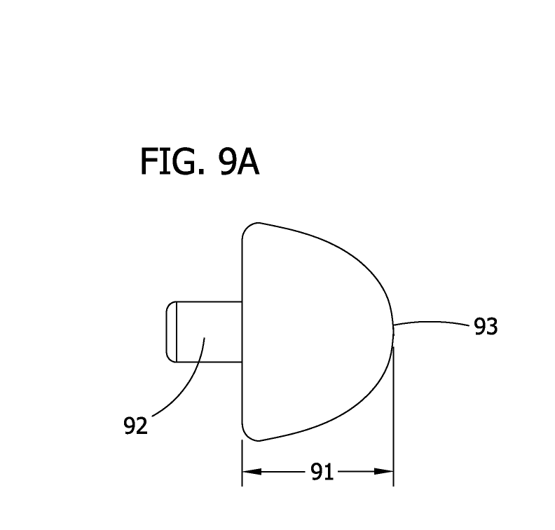

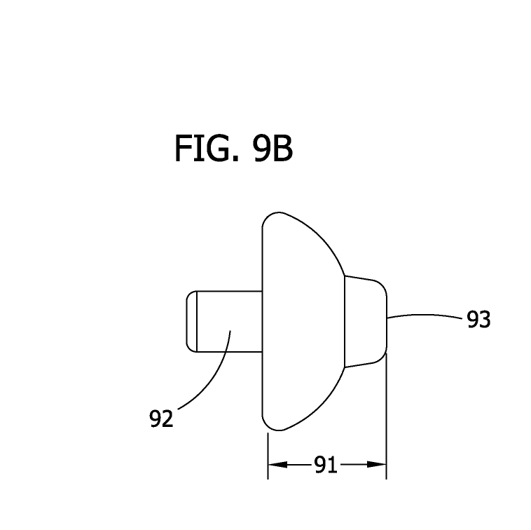

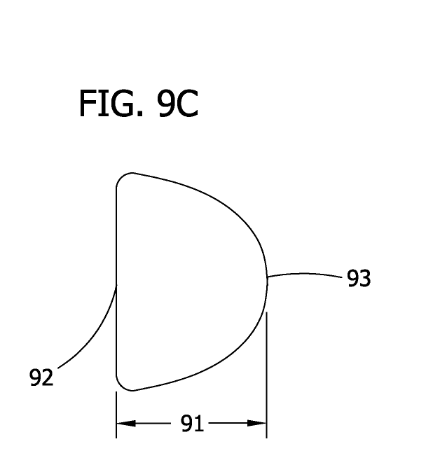

Referring to FIG. 9, the ear pad 87 may comprise a forward portion 91 that contacts the auditory meatus or tragus of the user, as previously described, and a rear portion 92 that connects the ear pad 87 with the extension member 90. The ear pad 87 may have a variety of shapes. Typically, the forward portion 91 of the ear pad 87 comprises a generally rounded or conical tapering shape, and terminates in a bluntly rounded tip 93. In one embodiment, the forward portion 91 of the ear pad 87 may be generally shaped to fit into the external auditory meatus.

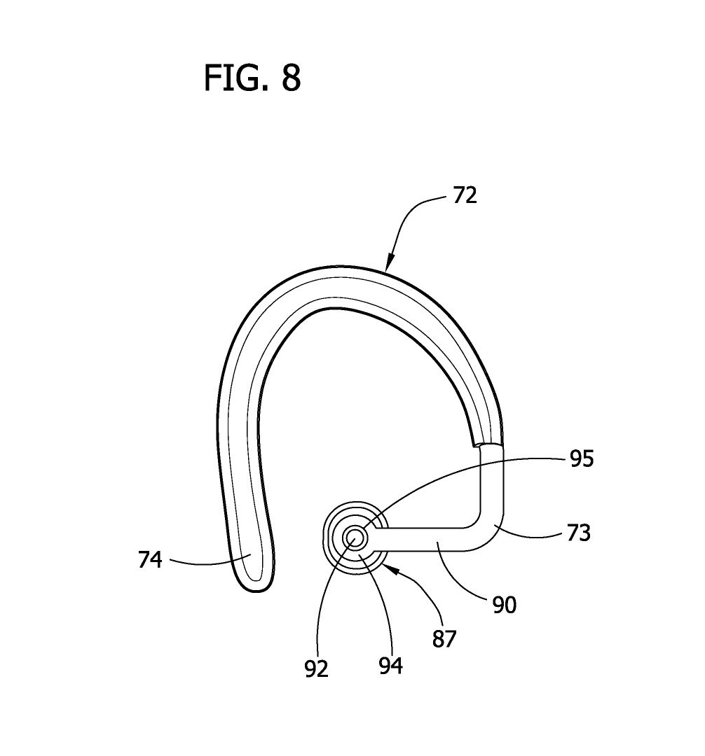

The ear pad 87 may be attached to the extension member 90 by any suitable means. Referring to FIG. 8, in one embodiment, the extension member 90 may comprise a generally circular flange 94 comprising a through hole 95 through which the rear portion 92 of the ear pad 87 extends. In this embodiment, the through hole 95 may have a diameter slightly smaller than the diameter of the rear portion 92 of the ear pad 87, so that the flange 94 presses against the sides of the rear portion 92, holding the ear pad 87 in place. In another embodiment, the ear pad 87 is attached to the extension member 90 using an adhesive (not shown). In this embodiment, the extension member 90 need not comprise the generally circular flange 94, and the rear portion 87 may optionally be relatively flat, as depicted in FIG. 9C.

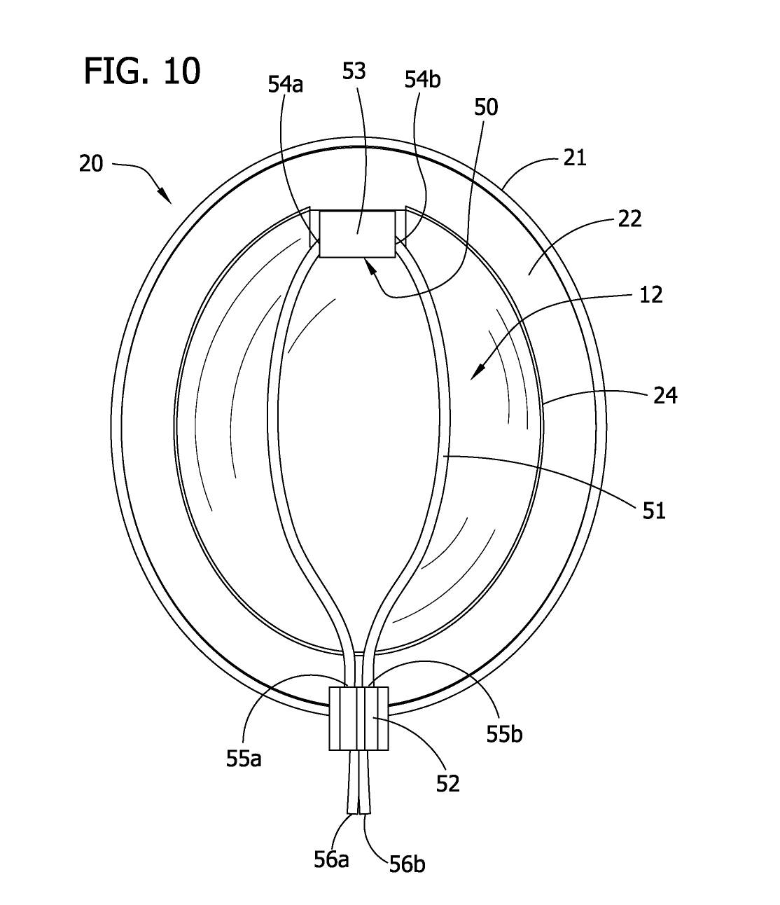

Referring now to FIGS. 10-12, there is depicted a hearing protection muff 10 comprising a self-attachment member that comprises a loop member 50, a flexible strap 51, and a fastener 52. The loop member 50 and the fastener 52 are attached to the rim portion 23 of the sound attenuating cap on generally opposite sides of the cavity 12. The loop member 50 comprises a closed channel 53 and a pair of apertures 54a, 54b, with one aperture at each end of the closed channel 53. Alternately, the loop member may be in the form of a ring member (not shown) comprising a single aperture through the ring.

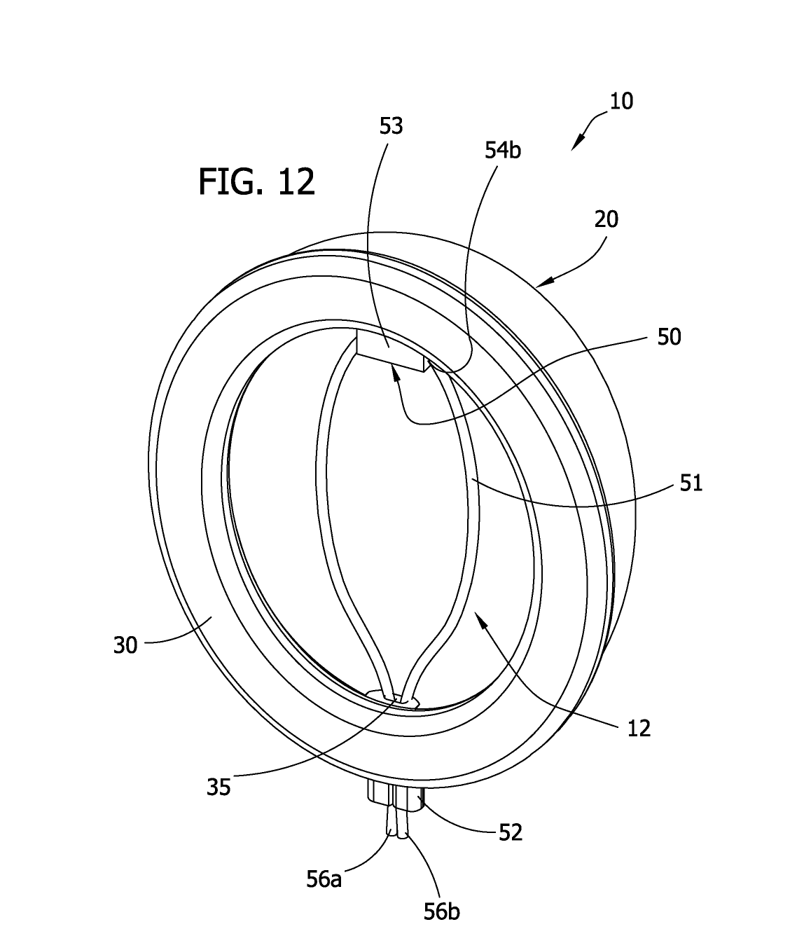

The self-attachment member further comprises a fastener 52 attached to the rim portion 23 of the sound attenuating cap 20 on the side of the cavity 12 generally opposite the loop member 50. The fastener 52 may comprise a pair of apertures 55a, 55b, as illustrated in FIG. 10, or alternately, may comprise a single aperture 35 as illustrated in FIG. 12. The fastener may be formed of any suitable material, such as, for example, plastics like polyethylene, polypropylene, polyvinyl chloride, and the like.

The flexible strap 51 acts in combination with the loop member 50 and the fastener 52 to secure the muff 10 to the ear of the user. Specifically, the flexible strap 51 passes through the aperture(s) of the loop member 50 and stretches across the cavity 12, where the end portions 56a, 56b of the strap 51 extend through the aperture(s) in the fastener 52. The flexible strap may be slidable through the aperture(s) in the loop member 50 and fastener 52. The end portions 56a, 56b of the flexible strap 51 are clamped together using the fastener 52, which may comprise a barrel cinch, clasp, or other mechanism which can hold the end portions 56a, 56b of the flexible strap 51 firmly with respect to each other, so that the strap 51 can be tightened. The fastener 52 is adjustable so that it can be manipulated to either release the end portions 56a, 56b or grasp them, as needed. The flexible strap 51 is thus adjustable between a loosened position and a tightened position, depending on the position of the fastener 52 on the strap 51. To attach the muff 10 to the ear, the user may slip the loosened strap around behind the ear, and then slide the fastener 52 up the strap 51, or pull the end portions 56a, 56b of the strap 51 through the fastener, tightening the strap, thus adjusting the diameter of the cavity 12 and securing the muff 10 to the ear. To remove the muff 10, the user slides the fastener 52 down the strap 51 towards the end portions 56a, 56b, thus loosening the strap 51, allowing removal of the muff 10 from the ear.

Typically, the flexible strap 51 is formed from a relatively soft, pliable material, such that the strap may stretch behind the ear of the user to comfortably secure the muff to the ear. Examples of suitable materials from which the flexible strap can be made include, for example, silicone, cotton string, polyvinyl chloride, neoprene, polypropylene, and the like. Loop member 53 may be formed from similar materials.

It should be noted that although FIG. 10 illustrates the flexible strap as comprising two different end portions 56a, 56b, each extending through a different aperture 55a, 55b in the fastener 52, in an alternate embodiment, the fastener may comprise a single aperture with both end portions 56a, 56b extending through the single aperture in the fastener 52, as illustrated in FIG. 12. In still another embodiment, the flexible strap may be in the form of a single, continuous loop comprising one end portion that passes through one or more of the aperture(s) of the fastener 52 (not shown).

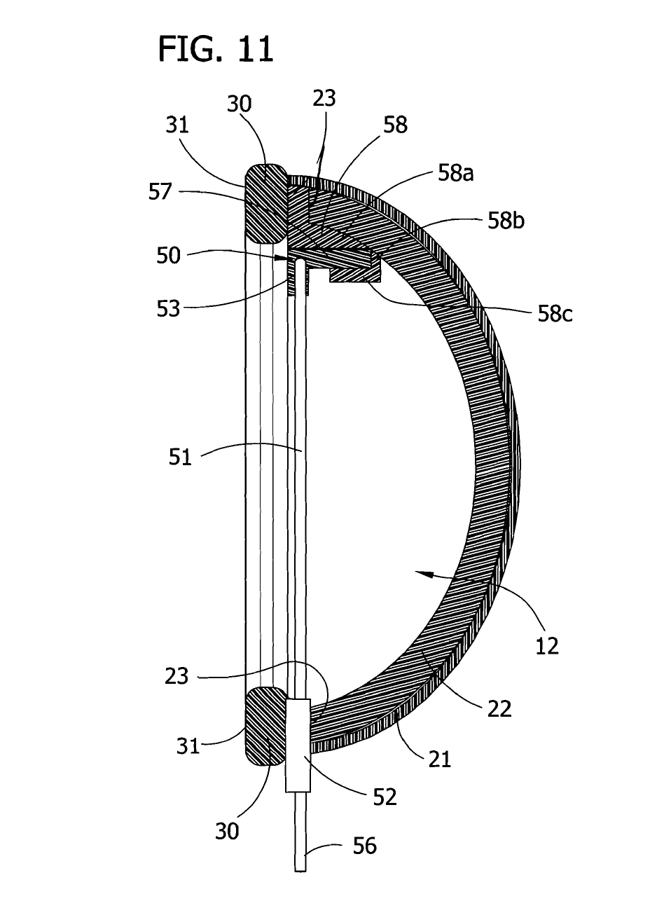

As noted above, the loop member is typically attached to the rim portion 23 of the sound attenuating cap 20 along the inner circumference 24. Any suitable attachment means may be used, such as, for example, adhesives. One particular means for attaching the loop member 50 to the rim portion 23 of the sound attenuating cap 20 is illustrated in FIG. 11. In this embodiment, the loop member 50 comprises a flange 57 that extends perpendicularly from the closed channel 53 to form a general “L” shape. An annular member 58 of relatively inflexible material, for example plastics such as polyethylene, polypropylene, polyvinyl chloride, and the like, is adhered to the rim portion 23 of the sound attenuating cap 20 using any suitable means, such as adhesive. Alternately, the annular member 58 may be an extension of the outer layer 21 of the sound attenuating cap 20, and thus may comprise sound reflecting material, as described above. The annular member extends around the rim portion 23 and has a portion 58a which is adhered to the cavity 12 facing side of the inner layer 22. As shown in FIG. 11, the annular member is shaped to provide a flange portion 58c which extends under flange 57 and a portion 58b which extends from the inner layer 22 and around the end of flange 57, connecting with flange portion 58c. The annular member 58 thus engages flange 57 to secure the loop member 50 to the sound attenuating cap 20. In one embodiment, the annular member 58 and the flange 57 may optionally also be bonded together for example, by ultrasonic bonding or adhesives. In another embodiment, the annular member 58 and the flange 57 may in fact be a single unit made of the same material (not shown), with the unit bonded to the sound attenuating cap using adhesive, or other suitable bonding means. It is to be understood that other means for attaching the loop member to the sound attenuating cap different from those illustrated and described herein are contemplated and are within the scope of the present disclosure.

The fastener 52 is attached to the rim portion 23 of the sound attenuating cap 20 using any suitable means such as, for example, adhesives.

As noted above, the muff 10 may further comprise a sealing member 30, which may be disposed adjacent to the rim portion 23 of the sound attenuating cap 20. When used in combination with a self-attachment member comprising a loop member, a flexible strap, and a fastener, as illustrated in FIG. 10-12, the sealing member 30 may overlie the annular member 58 and the fastener 52, thus providing a comfortable fit of the muff against the head of the user.

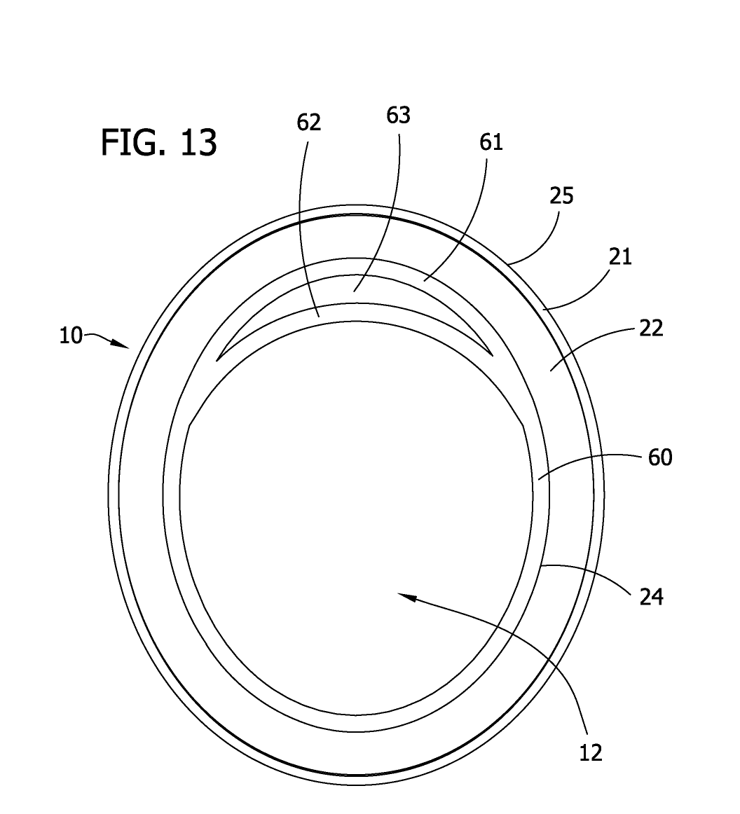

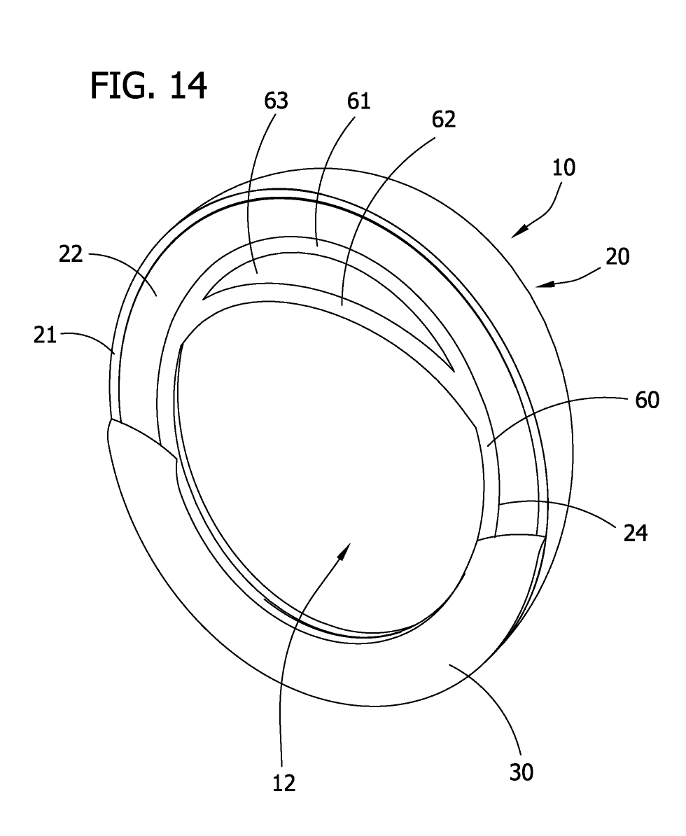

Referring now to FIGS. 13 and 14, there is depicted a hearing protection muff 10 comprising a self-attachment member that comprises a strap 60 attached to the rim portion of the sound attenuating cap 20 along the inner circumference 24. The strap 60 comprises an opening 63 along at least a portion of the strap 60. A first portion 61 of strap 60 along the opening 63 is attached to the rim portion 23 along the inner circumference 24, while a second portion 62 of strap 60 along the opening 63 is unattached. The unattached portion 62 of the strap 60 is capable of wrapping behind at least a portion of the pinna of the ear of the user.

To attach the muff 10 to the ear of a user, the user may stretch the unattached portion 62 of the strap 60 around at least a portion of the pinna of the ear to secure the muff 10 to the ear. The unattached portion 62 may wrap around the entire pinna of the ear, or alternately, may wrap around a portion of the pinna. For example, as illustrated in FIGS. 13 and 14, the opening 63 may run through the top half to top third of strap 60. The unattached portion 62 of the strap 60 may thus wrap around the top portion of the ear, holding the muff 10 in place over the ear. Alternately, the opening may run through up to about 75% of the strap, thus creating an opening in the strap large enough so that the unattached portion of the strap may wrap around the entire pinna of the ear (not shown).

The strap 60 is typically formed of a flexible material. Preferably, the strap is elastic in nature, so that the unattached portion 62 may be stretched to wrap around the pinna of the ear. Examples of suitable materials from which the strap 60 can be made include, for example, silicone, polyvinyl chloride, cotton string, and the like.

The strap 60 is typically attached to the rim portion of the sound attenuating cap 20 along the inner circumference 24, as illustrated in FIGS. 13 and 14. Any suitable attachment means may be used, such as, for example, using adhesives.

As noted above, the muff 10 may further comprise a sealing member 30, which has a surface for being placed against a portion of the user's head. When used in combination with a self-attachment member comprising a strap 60 attached to the rim portion of the sound attenuating cap 20 along the inner circumference 24, as illustrated in FIGS. 13 and 14, the sealing member 30 may be adjacent to all or a portion of the attached strap 60 and rim portion. In one embodiment, the sealing member 30 is disposed adjacent to the self-attachment member, e.g., the attached strap 60, and rim portion along the lower half to lower third of the muff, as illustrated in FIG. 14. In other embodiments, the sealing member may be disposed along the entire attached portion of the strap and the corresponding portion of the rim portion (not shown).

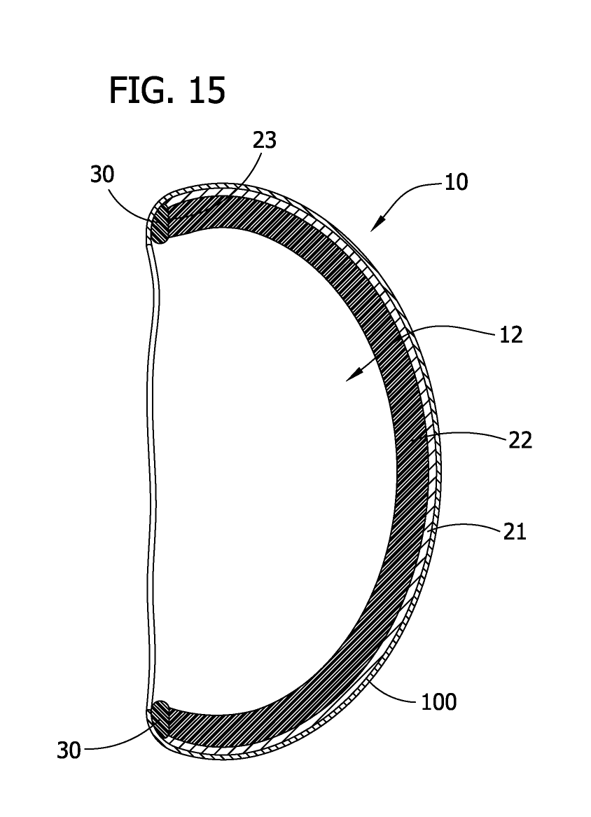

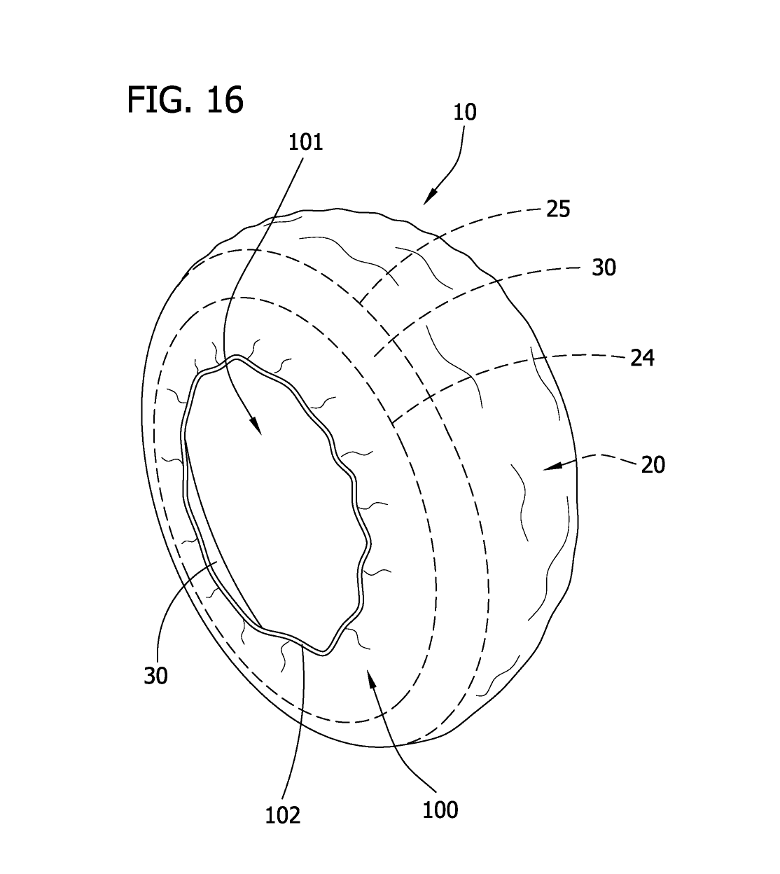

In another embodiment, the self-attachment member may comprise a covering material that can stretch to fit various sizes of ears. For instance, referring now to FIGS. 15 and 16, there is depicted a hearing protection muff 10 comprising a self-attachment member that comprises a covering material 100 disposed adjacent to the outer layer 21 of the sound attenuating cap 20, and encompassing the entirety of the sound attenuating cap 20. The edges of the covering material 100 extend around the rim portion 23 of the sound attenuating cap, forming an opening 101 through which the ear is placed when the muff 10 is in use. The opening 101 formed by the edges of the covering material 100 typically has a smaller circumference than the inner circumference 24 that forms the inner edge of the rim portion of the sound attenuating cap 20. The edges of the covering material 100 may optionally have attached thereto an elastic member 102 that pulls the edges of the covering material 100 around the rim portion. To engage the sound attenuating cap 20 to the ear of the user, the elastic member 102 is stretched over the ear as the ear is placed through the opening 101 formed by the edges of the covering material 100. The elastic member 102 pulls the edges of the covering material 100 securely around the rim portion of the sound attenuating cap 20, securing the muff 10 to the ear of the user.

Alternately, the covering material 100 itself may be formed from an elastic material. In this embodiment, it is not necessary for the edges of the covering material 100 to have attached thereto an elastic member 102. In this embodiment, the sound attenuating cap 20 is engaged to the ear of the user by stretching the elastic covering material 100 over the ear as the ear is placed through the opening 101 formed by the edges of the covering material 100. The elasticity of the covering material holds the sound attenuating cap 20 securely over the ear of the user.

As noted above, the covering material 100 is disposed adjacent to the outer layer of the sound attenuating cap 20, and encompasses the sound attenuating cap 20. In certain embodiments, the covering material 100 may be securely attached to the outer layer of the sound attenuating cap using any suitable means, such as adhesives. In alternate embodiments, the covering material 100 is not attached to the sound attenuating cap, but rather, is held in place around the sound attenuating cap only by the pull of the elastic member 102 on the edges of the covering material 100, or alternately, by the elasticity of the covering material, as previously described.

As noted above, the covering material 100 and/or the elastic member 102 may be formed from a soft elastic material, capable of stretching over the exterior of the sound attenuating cap and around the ear of the user. Examples of suitable materials from which the covering material and/or elastic member can be made include, for example, flexible vinyl, silicone, and the like. Alternately, the covering material may be formed of any cloth or plastic material. In this embodiment, it is preferable for the covering material to have an elastic member 102 attached to its edges, to hold the covering material in place, as previously described.

As noted above, the muff 10 may further comprise a sealing member 30, which may be disposed adjacent to the rim portion 23 of the sound attenuating cap 20. When used in combination with a self-attachment member comprising the covering material 100, as illustrated in FIGS. 15 and 16, the sealing member 30 may overlie all or a portion of the rim portion of the sound attenuating cap 20, thus providing a secure and comfortable fit of the muff 10 against the head of the user.

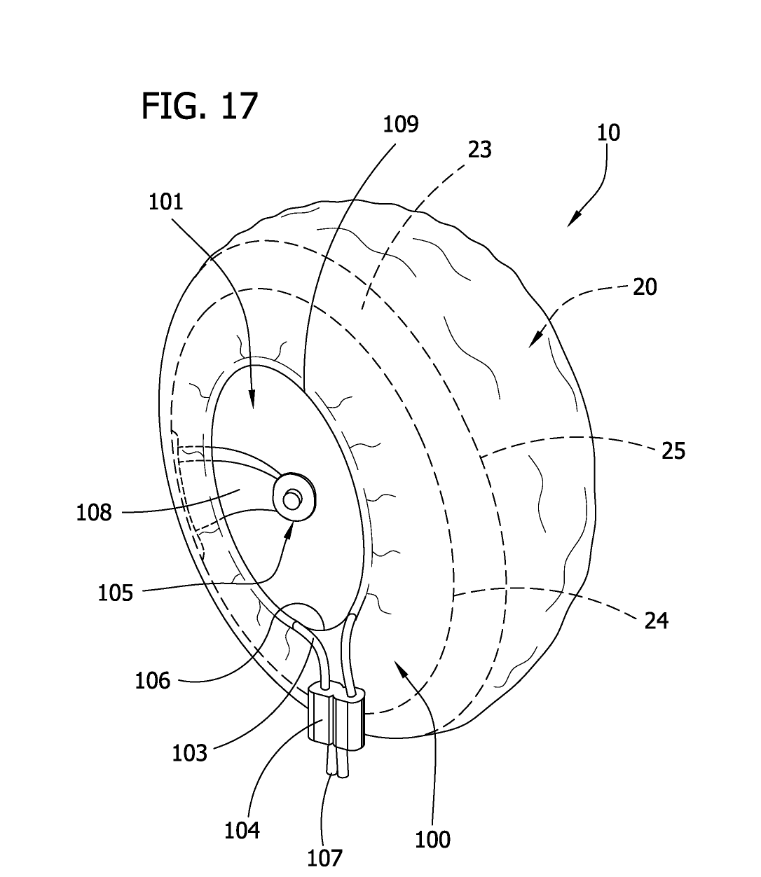

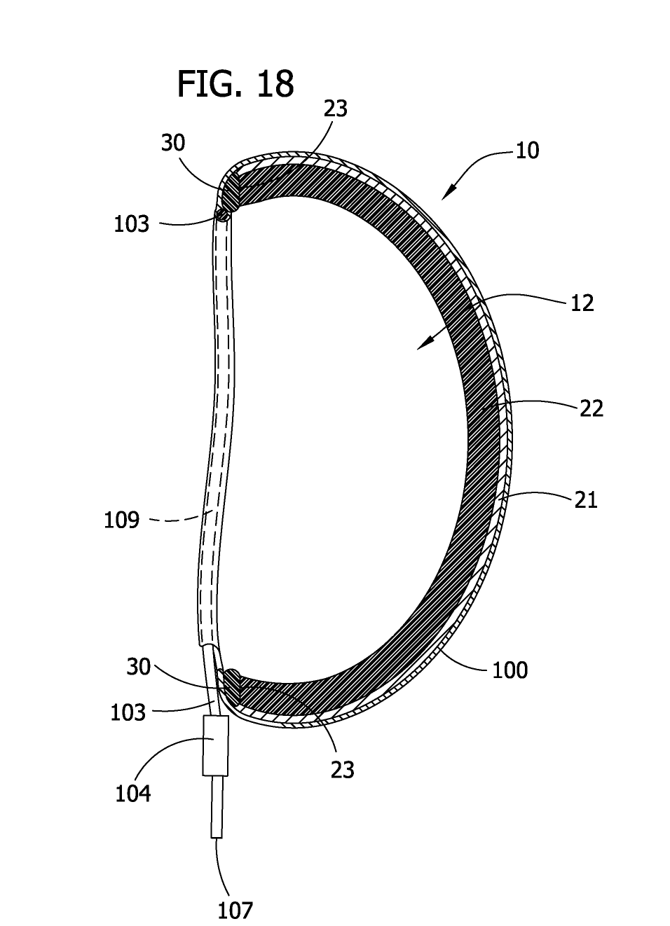

Referring now to FIGS. 17 and 18, there is depicted a hearing protection muff 10 comprising a self-attachment member that comprises a covering material 100, a flexible strap 103, a fastener 104, and an ear pad 105. The covering material 100 is disposed adjacent to the outer layer of the sound attenuating cap 20 and encompasses the entirety of the sound attenuating cap 20. The edge 106 of the covering material 100 extends around the rim portion 23 of the sound attenuating cap 20, forming an opening 101 through which the ear is placed when the muff 10 is in use. The opening 101 formed by the edges of the covering material 100 typically has a smaller circumference than the inner circumference 24 that forms the inner edge of the rim portion 23 of the sound attenuating cap 20. The edge of the covering material 100 forms a closed channel 109 along at least a portion thereof. The flexible strap 103 is located within the closed channel 109 and comprises at least one end portion 107 outside the closed channel. The flexible strap 103 is slidable with in the closed channel 109 so that when used in combination with the fastener 104, the flexible strap 103 can be adjusted between a loosened position and a tightened position. When tightened, the flexible strap 103 pulls the edges of the covering material 100 around the rim portion, to secure the sound attenuating cap 20 to the ear of the user.

As noted above, the flexible strap 103 may be used in combination with the fastener 104 to tighten the covering material 100 around the sound attenuating cap 20, thus securing the sound attenuating cap 20 to the ear of the user. More particularly, the flexible strap 103 comprises an end portion 107 outside the closed channel 109 that extends through a pair of apertures in the fastener 104. Alternately, the fastener 104 may comprise a single aperture through which the end portion 107 of the flexible strap 103 may extend.

The end portion 107 of the flexible strap 103 is clamped together using the fastener 104, which may comprise a barrel cinch, clasp, or other mechanism which can hold the end portion 107 of the flexible strap firmly, so that the strap 103 can be tightened. The fastener 104 is adjustable so that it can be manipulated to either release the end portion 107 or grasp it as needed. The flexible strap 103 is thus adjustable between a loosened position and a tightened position, depending on the position of the fastener 104 on the strap 103. To attach the muff 10 to the ear, the user may slip the loosened strap around behind the ear, and then slide the fastener 104 up the strap 103, or pull the end portion 107 of the strap 103 through the fastener, tightening the strap 103, which acts to pull the covering material 100 securely around the sound attenuating cap 20, securing the muff 10 to the ear of the user. To remove the muff 10, the user slides the fastener 104 down the strap 103 towards the end portion 107, thus loosening the strap 103, and subsequently the covering material 100, allowing removal of the muff 10 from the ear.

Typically, the flexible strap 103 is formed from a relatively elastic material, such that the strap may stretch behind the ear of the user to comfortably secure the muff to the ear. Examples of suitable materials from which the flexible strap can be made include, for example, silicone, cotton string, neoprene, polypropylene, and the like.

It should be noted that although FIG. 17 illustrates the flexible strap as comprising a pair of end portions 107 that extend through a pair of apertures in the fastener 104, in an alternate embodiment, the end portions 107 of the flexible strap 103 may extend through a single aperture in the fastener. In still another embodiment, the flexible strap may comprise a single end portion in the form of a continuous loop that extends through the aperture(s) in the fastener (not shown).

As noted above, the covering material 100 is disposed adjacent to the outer layer of the sound attenuating cap 20, and encompasses the sound attenuating cap 20. In certain embodiments, the covering material 100 may be securely attached to the outer layer of the sound attenuating cap using any suitable means, such as adhesives. In other embodiments, the covering material 100 is not attached to the sound attenuating cap, but rather, is held in place around the sound attenuating cap only by the pull of the flexible strap 103 on the edges of the covering material 100.

The covering material 100 is preferably formed from a soft elastic material, capable of stretching over the exterior of the sound attenuating cap and around the ear of the user. Examples of suitable materials from which the covering material can be made include, for example, flexible vinyl, silicone, and the like. Alternately, the covering material may be formed of any cloth or plastic material.

The self-attachment member further comprises an ear pad 105 attached to the rim portion of the sound attenuating cap 20 along the inner circumference 24. The ear pad may be attached to the inner circumference 24 using any suitable means. For instance, in one embodiment, an extension member 108 extends inwardly from the inner circumference 24 towards the center of the cavity 12, generally parallel to the plane defined by the rim portion 23 of the sound attenuating cap 20. The extension member 108 has attached thereto the ear pad 105. The extension member 108 may be attached to the inner circumference 24 by any suitable means including, for example, adhesives, ultrasonic bonding, pressure bonding, and the like. The exact size and shape of the extension member 108 is not critical, but it is preferable that the extension member extend inwardly from the inner circumference 24 generally far enough so that the ear pad 105 is positioned over the external auditory meatus when the muff 10 is worn. The ear pad 105 thus positioned acts to block out sound, improving the sound attenuating effect of the muff 10. In an alternate embodiment, the extension member 108 with attached ear pad 105 may be positioned such that the ear pad 105 overlies the tragus of the ear of a user when the muff 10 is worn, so that pressure exerted by the ear pad 105 when the muff 10 is worn will fold the tragus over the auditory canal of the user's ear to block out sound without the necessity of the ear pad 105 entering the auditory canal. The ear pad 105 may advantageously help improve the sound attenuating effects of the muff and help secure the muff to the user's ear.

The ear pad 105 may comprise an ear plug or other resilient member. Preferably, the ear pad is comprised of a resilient material such as, for example, foamed rubber, plastic, gel, silicone, and the like.

The ear pad 105 may be attached to the extension member 108 by any suitable means. In one embodiment, the extension member 108 may comprise a generally circular flange (not shown) comprising a through hole (not shown) through which the rear portion of the ear pad 105 extends. This attachment means is similar to that illustrated in FIGS. 8 and 9, previously described. In another embodiment, the ear pad 105 is attached to the extension member 108 using an adhesive (not shown).

As noted above, the muff 10 may further comprise a sealing member 30, which may be disposed adjacent to the rim portion 23 of the sound attenuating cap 20. When used in combination with a self-attachment member comprising a covering material 100, a flexible strap 103, a fastener 104, and an ear pad 105, as illustrated in FIGS. 17 and 18, the sealing member may optionally overlie all or a portion of the rim portion of the sound attenuating cap 20, thus providing a comfortable fit of the muff against the head of the user.

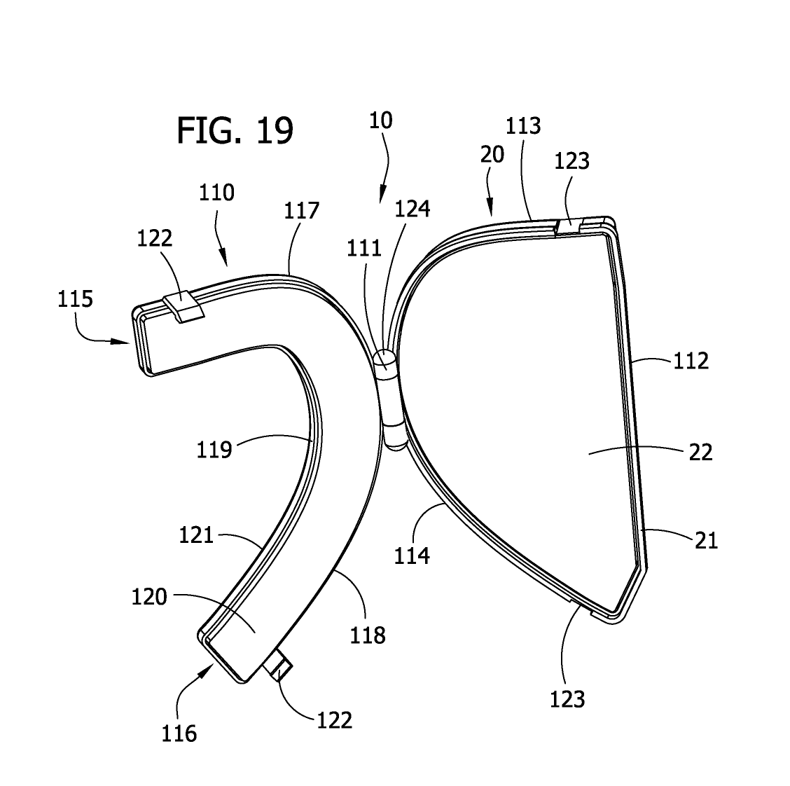

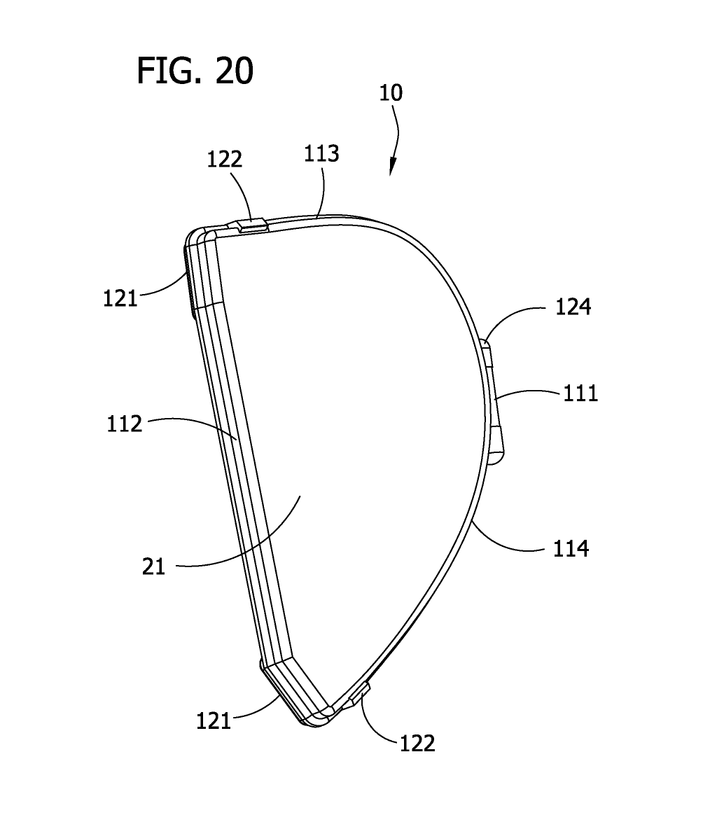

Referring now to FIGS. 19 and 20, there is depicted another configuration of a muff of the present disclosure. In this embodiment, the muff 10 comprises a sound attenuating cap 20 and a self-attachment member comprising an ear clip 110, a hinge 111, and a snapping mechanism. The sound attenuating cap 20 comprises an outer layer 21 that comprises a sound reflecting material and an inner layer 22 that comprises a sound absorbing material, as previously described. The inner layer 22 forms the inner surface of the hearing protection muff 10 that faces the ear of the user. The inner layer 22 is disposed adjacent to the outer layer 21 and is uniformly bonded thereto. The outer layer 21 forms the outer surface of the sound attenuating cap 20 that faces away from the ear of the user. The sound attenuating cap 20 may be domed or flat, but in this embodiment is typically flat, such that the inner layer 22 contacts the front portion of the pinna of the ear when the muff 10 is worn.

The sound attenuating cap 20 is generally ear shaped. For instance, the sound attenuating cap 20 has a body facing edge 112, an upper edge 113, and a curved edge 114. When the muff 10 is being worn, the body facing edge 112 is positioned near the base of the ear of the user along the side of the user's head, the upper edge 113 runs along the top portion of the pinna of the ear, and the curved edge 114 runs along the exterior curved edge of the pinna of the ear.

The sound attenuating cap 20 is engaged to the ear of the user by means of a self-attachment member comprising a hinge 111, an ear clip 110, and a snapping mechanism. The ear clip 110 comprises a first end portion 115 and a second end portion 116 spaced apart from the first end portion to form a substantially “U” shape, as illustrated in FIG. 19. The ear clip 110 has an upper edge 117, an exterior curved edge 118, and an inner curved edge 119. It should be understood that the exact shape of the ear clip 110 and the distance between the first end portion 115 and the second end portion 116 is not critical, but should generally be such that when the muff is being worn, the exterior curved edge 118 of the ear clip conforms to the curved edge 114 of the sound attenuating cap, and the upper edge 117 of the ear clip conforms to the upper edge 113 of the sound attenuating cap.

When the muff is worn, the ear clip 110 is positioned behind the pinna of the ear of a user. The ear clip 110 may thus comprise an inner ear clip layer 120 that is in contact with the back of the pinna of the user when the muff is worn, and an outer ear clip layer 121 that faces away from the pinna of the user. Because the inner ear clip layer 120 is in contact with the pinna of the user when the muff is worn, in one embodiment, the inner ear clip layer may comprise a relatively soft, cushioning material, such as, polyurethane foam, ethylene-vinyl acetate foam, polyolefin fibers, and the like. In one embodiment, the inner ear clip layer 120 may comprise sound absorbing materials, such as those used to form the inner layer 22 of the sound attenuating cap 20.

The outer ear clip layer 121 forms the external shell of the ear clip, and thus typically comprises a more rigid material. For instance, in one embodiment, the outer ear clip layer 121 may comprise sound reflecting materials, such as those used to form the outer layer 21 of the sound attenuating cap 20.

The ear clip 110 is attached the sound attenuating cap 20 by means of a hinge 111. The hinge 111 is typically located along the curved edge 114 of the sound attenuating cap 20 and the exterior curved edge 118 of the ear clip 110. A hinge pin 124 joins the outer ear clip layer 121 and the outer layer 21 of the sound attenuating cap 20 in a rotating connection that allows the ear clip 110 to freely rotate with respect to the sound attenuating cap 20.

Referring again to FIG. 19, to engage the muff 10 to the ear of the user, the user places the inner layer 22 of the sound attenuating cap 20 against the front of the pinna, and applies force to the outer layer 121 of the ear clip 110, progressively pivoting the ear clip 110 towards the back portion of the pinna, until the inner layer 120 of the ear clip 110 is pressed against the back of the pinna. The ear clip 110 may then be securely connected to the sound attenuating cap 20 by means of the snapping mechanism.

More particularly, the snapping mechanism comprises at least two snap catches 122 on the ear clip 110. The exact location of the snap catches on the ear clip 110 is not critical, but typically one snap catch will protrude from the upper edge 117 of the ear clip 110 and another will protrude from the exterior curved edge 118 of the ear clip 110. Preferably, one snap catch 122 is located towards the first end portion 115, while the other snap catch 122 is located towards the second end portion 116.

The snapping mechanism further comprises at least two grooves 123 for receipt of the snap catches. As illustrated in FIG. 19, one groove 123 is located along the upper edge 113 of the sound attenuating cap 20, while the other groove 123 is located along the curved edge 114 of the sound attenuating cap 20. The positioning of the grooves 123 along the upper edge 113 and curved edge 114 of the sound attenuating cap 20 should be such that the grooves 123 align with the snap catches 122 protruding from the ear clip 110, when the muff 10 is attached to the ear.

To engage the snapping mechanism, the ear clip 110 is pushed towards the sound attenuating cap 20 until the snap catches 122 protruding from the ear clip 110 lock into the grooves 123 on the sound attenuating cap 20. The snap catches 122 may have a resilient structure that enables the snap catches 122 to be deformed as they are pressed into the grooves 123, but resiliently return to their natural position when interlocked with the grooves 123. The muff 10 may be removed from the ear of a user by pulling the ear clip 110 and sound attenuating cap 20 apart, disengaging the snapping mechanism.

In another embodiment, the muff 10 may comprise a sound attenuating cap 20 and a self-attachment member comprising an ear clip 110 and a hinge 111 comprising a torsion spring. In this embodiment, the muff 10 is substantially the same as the muff depicted in FIG. 19, except instead of a snapping mechanism comprising snap catches 122 and grooves 123, the muff comprises a hinge 111 with a torsion spring (not shown). The torsion spring collapses the ear clip 110 and the sound attenuating cap 20 together, thus clamping the muff 10 onto the ear of a user. Thus, to engage the muff 10 to the ear, a user pulls the ear clip 110 and the sound attenuating cap 20 apart and places the inner layer 22 of the sound attenuating cap 20 against the front of the pinna. Force exerted by the torsion spring will automatically pivot the ear clip 110 towards the back portion of the pinna, securely clamping the muff 10 onto the ear. It should be understood that numerous configurations of torsion springs are known in the art and may be used in the muffs described herein.

It is to be understood that the self-attachment members described herein are illustrative, and other self-attachment mechanisms and configurations may also be used in the muffs of the present disclosure.

Having described the disclosure in detail, it will be apparent that modifications and variations are possible without departing from the scope of the disclosure defined in the appended claims.

When introducing elements of the present disclosure or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved and other advantageous results attained.

As various changes could be made in the above products without departing from the scope of the disclosure, it is intended that all matter contained in the above description and shown in the accompanying drawings shall be interpreted as illustrative and not in a limiting sense.

What is claimed is:

1. A bandless, hearing protection muff comprising:
a sound attenuating cap defining a cavity for covering an external ear of a user, the sound attenuating cap having a rim portion for encircling the ear;
a sealing member having a surface for being placed against a portion of the user's head; and
a self-attachment member for engaging the sound attenuating cap to the ear of the user;
wherein the sound attenuating cap comprises:
an outer layer comprising a sound reflecting material, the sound reflecting material having a sound absorption coefficient of no more than about 0.4 at a frequency of from about 800 Hz to about 10,000 Hz, and
an inner layer disposed adjacent to the outer layer, the inner layer comprising a sound absorbing material, the sound absorbing material having a sound absorption coefficient of at least about 0.6 at a frequency of from about 800 Hz to about 10,000 Hz,
wherein the sound attenuating cap attenuates sound at from about 8 dB to about 33 dB over the frequency range of from about 125 Hz to about 8,000 Hz.

2. The muff of claim 1 wherein the sealing member is selected from the group consisting of polyurethane foam, ethylene-vinyl acetate, silicone rubber, ethylene propylene rubber, a contact adhesive, a silicone adhesive, a hydrogel adhesive, and combinations thereof.

3. The muff of claim 1 wherein the sealing member is disposed adjacent to the rim portion of the sound attenuating cap.

4. The muff of claim 1 further comprising an adhesive on the surface of the sealing member.

5. The muff of claim 4 wherein the adhesive is selected from the group consisting of silicone adhesives, hydrogel adhesives, pressure sensitive adhesives, and combinations thereof.

6. The muff of claim 1 wherein the sound reflecting material is selected from the group consisting of mass loaded vinyl, high density plastic, metal, wood, and combinations thereof.

7. The muff of claim 1 wherein the sound absorbing material is selected from the group consisting of glass fiber, fibrous mineral wool, open cell foam, semi-open cell foam, and combinations thereof.

8. The muff of claim 1 wherein the outer layer has a thickness of from about 1 millimeter to about 10 millimeters.

9. The muff of claim 1 wherein the inner layer has a thickness of from about 5 millimeters to about 40 millimeters.

10. The muff of claim 1 wherein the outer layer comprising a sound reflecting material having a sound absorption coefficient of no more than about 0.2 at a frequency of from about 800 Hz to about 10,000 Hz.

11. The muff of claim 1 wherein the inner layer comprises a sound absorbing material having a sound absorption coefficient of at least about 0.8 at a frequency of from about 800 Hz to about 10,000 Hz.

12. The muff of claim 1 wherein the self-attachment member comprises a loop member, a flexible strap, and a fastener, the loop member and the fastener being attached to the rim portion of the sound attenuating cap on opposite sides of the cavity, the loop member comprising at least one aperture through which the flexible strap passes, the fastener comprising at least one aperture, and the flexible strap comprising at least one end portion which extends through the at least one aperture in the fastener, the flexible strap being slidable through the at least one aperture in the fastener so that the flexible strap is adjustable between a loosened position and a tightened position to adjust the diameter of the cavity and secure the sound attenuating cap to the ear of the user.

13. The muff of claim 1 wherein the rim portion has a thickness defined by an inner circumference and an outer circumference and the self-attachment member comprises a strap, the strap being attached to the rim portion along the inner circumference, the strap comprising an opening along at least a portion of the strap, wherein a first portion of the strap along the opening is attached to the rim portion along the inner circumference and a second portion of the strap along the opening is unattached, the unattached portion of the strap being capable of wrapping behind at least a portion of the pinna of the ear of the user.

14. The muff of claim 1 wherein the self-attachment member comprises a covering material disposed adjacent to the outer layer of the sound attenuating cap and encompassing the sound attenuating cap, and an elastic material attached to the edges of the covering material, wherein the elastic material pulls the edges of the covering material around the rim portion to secure the sound attenuating cap to the ear of the user.

15. The muff of claim 1 wherein the self-attachment member comprises an elastic covering material disposed adjacent to the outer layer of the sound attenuating cap and encompassing the sound attenuating cap, wherein the elastic covering material secures the sound attenuating cap to the ear of the user.

16. The muff of claim 1 wherein the rim portion has a thickness defined by an inner circumference and an outer circumference, and the self attachment member comprises an ear pad attached to the rim portion along the inner circumference, a covering material disposed adjacent to the outer layer of the sound attenuating cap and encompassing the sound attenuating cap, wherein the edges of the covering material form a closed channel along at least a portion thereof, a flexible strap located within the closed channel and comprising at least one end portion outside the closed channel, and a fastener comprising at least one aperture, wherein the at least one end portion of the flexible strap extends through the at least one aperture in the fastener, the flexible strap being slidable within the closed channel and through the at least one aperture in the fastener so that the flexible strap is adjustable between a loosened position and a tightened position.

17. A bandless, hearing protection muff comprising:
a sound attenuating cap defining a cavity for covering an external ear of a user, the sound attenuating cap having a rim portion for encircling the ear;
a self-attachment member for engaging the sound attenuating cap to the ear of the user, the self-attachment member comprising:
a substantially U-shaped ear clip providing a first end portion and a second end portion spaced from the first end portion, and
a rigid frame comprising a hinge, the hinge comprising a torsion spring, the rigid frame being disposed adjacent to the rim portion of the sound attenuating cap,
wherein the first end portion of the ear clip extends through the hinge, and the second end portion of the ear clip is capable of wrapping behind the pinna of the ear of the user; and
a sealing member disposed adjacent to the rigid frame, the sealing member having a surface for being placed against a portion of the user's head;
wherein the sound attenuating cap comprises:
an outer layer comprising a sound reflecting material, the sound reflecting material having a sound absorption coefficient of no more than about 0.4 at a frequency of from about 800 Hz to about 10,000 Hz, and
an inner layer disposed adjacent to the outer layer, the inner layer comprising a sound absorbing material, the sound absorbing material having a sound absorption coefficient of at least about 0.6 at a frequency of from about 800 Hz to about 10,000 Hz,
wherein the sound attenuating cap attenuates sound at from about 8 dB to about 33 dB over the frequency range of from about 125 Hz to about 8,000 Hz.

18. The muff of claim 17 wherein the self-attachment member further comprises an ear pad attached to the first end portion.

19. A bandless, hearing protection muff comprising:
a sound attenuating cap for covering an external ear of a user; and
a self-attachment member for engaging the sound attenuating cap to the ear of the user, the self-attachment member comprising:
an ear clip comprising a first end portion and a second end portion spaced from the first end portion,
a hinge for attaching the ear clip to the sound attenuating cap, and
a snapping mechanism,
wherein the sound attenuating cap comprises:
an outer layer comprising a sound reflecting material, the sound reflecting material having a sound absorption coefficient of no more than about 0.4 at a frequency of from about 800 Hz to about 10,000 Hz, and
an inner layer disposed adjacent to the outer layer, the inner layer comprising a sound absorbing material, the sound absorbing material having a sound absorption coefficient of at least about 0.6 at a frequency of from about 800 Hz to about 10,000 Hz,
wherein the sound attenuating cap attenuates sound at from about 8 dB to about 33 dB over the frequency range of from about 125 Hz to about 8,000 Hz.

20. A bandless, hearing protection muff comprising:
a sound attenuating cap for covering an external ear of a user; and
a self-attachment member for engaging the sound attenuating cap to the ear of the user, the self-attachment member comprising:
an ear clip comprising a first end portion and a second end portion spaced from the first end portion; and
a hinge for attaching the ear clip to the sound attenuating cap, the hinge comprising a torsion spring,
wherein the sound attenuating cap comprises:
an outer layer comprising a sound reflecting material, the sound reflecting material having a sound absorption coefficient of no more than about 0.4 at a frequency of from about 800 Hz to about 10,000 Hz, and
an inner layer disposed adjacent to the outer layer, the inner layer comprising a sound absorbing material, the sound absorbing material having a sound absorption coefficient of at least about 0.6 at a frequency of from about 800 Hz to about 10,000 Hz,
wherein the sound attenuating cap attenuates sound at from about 8 dB to about 33 dB over the frequency range of from about 125 Hz to about 8,000 Hz.
